(19) United States
(12) Patent Application Publication
(10) Pub. No.: US 2011/0171277 A1
(43) Pub. Date: Jul. 14, 2011

(54) WOUND DRESSING HAVING A POLYURETHANE FOAM LAYER AND A COVER LAYER MADE OF THERMOPLASTIC POLYMER

(75) Inventor: Jan Schönberger, Solingen (DE)

(73) Assignee: Bayer MaterialScience AG, Leverkusen (DE)

(21) Appl. No.: 13/120,037

(22) PCT Filed: Sep. 8, 2009
(86) PCT No.: PCT/EP2009/006505

§ 371 Date: Mar. 21, 2011

(30) Foreign Application Priority Data
Sep. 19, 2008 (EP) 08164722.4

(51) Int. Cl. A61K 9/00; A61K 8/02; A61P 17/02; B32B 37/16

(52) U.S. Cl. 424/401; 424/400; 156/60

(57) ABSTRACT

The present invention relates to a layered composite which includes a foam layer and a cover layer. The cover layer includes a thermoplastic polymer and is at least partly bonded directly to the foam layer. The foam layer includes a polyurethane foam obtained by frothing and drying a composition that includes an aqueous, anionically hydrophilicized polyurethane dispersion.

The present invention relates to a layered composite useful as wound dressing, comprising a foam layer and also a covering layer, wherein the covering layer comprises a thermoplastic polymer and is at least partly bonded directly to the foam layer. The present invention further relates to a process for producing such a layered composite and also to its use as a wound dressing for example.

Wound management may utilize wound dressings having a foam layer which lies on the wound. This has proved advantageous since the foam's ability to absorb moisture exuding from the wound creates a climate in the wound that is beneficial to healing. However, such foams per se often have the disadvantage that they can get dirty, colonized by bacteria or destroyed through mechanical stresses during wear.

As a remedy, a protective covering foil can be provided on the outside surface of a wound dressing. This covering foil can provide for a microbial impermeability, for an impermeability to wound exudate coupled with simultaneous permeability of the wound dressing to water vapour. Hitherto the covering foil has to be bonded to the foam layer by means of an adhesive.

The use of an adhesive has disadvantages, however. Applying a layer of adhesive represents additional labour and material requirements and hence additional costs in the fabrication of a wound dressing. Moreover, the adhesive can have an unfavourable effect on the permeability to water vapour. Finally, the adhesive can also affect the thermal formability of the wound dressing.

WO 2007/115696, fully incorporated herein by reference, discloses a process for producing polyurethane foams for wound treatment wherein a composition comprising a polyurethane dispersion and specific coagulants is frothed and dried. The polyurethane dispersions are obtainable for example by preparing isocyanate-functional prepolymers from organic polyisocyanates and polymeric polyols having number average molecular weights of 400 g/mol to 8000 g/mol and OH functionalities of 1.5 to 6 and also optionally with hydroxyl-functional compounds having molecular weights of 62 g/mol to 399 g/mol and optionally isocyanate-reactive, anionic or potentially anionic and optionally nonionic hydrophilicizing agents. The free NCO groups of the prepolymer are then optionally reacted in whole or in part with amino-functional compounds having molecular weights of 32 g/mol to 400 g/mol and also with amino-functional, anionic or potentially anionic hydrophilicizing agents with chain extension. The prepolymers are dispersed in water before, during or after this step. Any potential ionic groups present are converted into the ionic form by partial or complete reaction with a neutralizing agent.

EP 0 485 657 discloses a dressing for wounds or dermal ulcers. The dressing includes a semipermeable polyurethane film and a plurality of concentric polyethylene foam rings. Application of foam rings to a semipermeable thin film permits modification of the moisture/vapour transmission properties of the wound dressing. These can be adjusted to suit the wound environment. The semipermeable thin film is bonded to the upper surface of a ring. It is further disclosed that this bond is achieved by means of an adhesive.

DE 103 01 835 discloses a plaster having a printed wound dressing and a transparent fixing foil. The wound dressing can be individually printed and, more particularly, be shaped having regard to the contours of printed motifs. The fixing foil adhered on one side of the wound-facing side overlaps the wound dressing on two sides at least and is of transparent material whereby the cosmetic impairment due to the plaster is essentially reduced to the size of the wound dressing. The superior tacky transparent fixing foil provides a firm hold even in problematic skin regions if additionally segmented. Also disclosed are a process for producing this plaster, its use and also methods of use. The transparent fixing foil can consist of polyurethane. According to this publication, the transparent fixing foil material is provided on the wound-facing side with a transparent adhesive which provides for fixing of the plaster to the skin and for fixing of the wound dressing to the fixing foil.

There is therefore a need for alternative wound dressings. More particularly, there is a need for wound dressings wherein a foam layer and a covering layer are bonded together without adhesive in a strength which does not limit the use and which are thermally formable.

The invention therefore proposes a layered composite useful as wound dressing, comprising a foam layer and also a covering layer, wherein the covering layer comprises a thermoplastic polymer and is at least partly bonded directly to the foam layer and wherein the foam layer comprises a polyurethane foam obtained by a composition comprising an aqueous, anionically hydrophilicized polyurethane dispersion (I) being frothed and dried.

The foam layer comprises a foam obtainable from a frothed polyurethane dispersion. It is this foam layer which is placed on the wound to be covered. Advantageously, this foam has a microporous, at least partly open-pore structure comprising intercommunicating cells.

The covering layer of the layered composite of the present invention comprises a thermoplastic polymer. Thermoplastic polymer is initially to be understood as meaning a polymer which remains thermoplastic when repeatedly heated and cooled in the temperature range typical for processing and using the material. Thermoplastic is to be understood as referring to the property of a manufactured polymer of, in a temperature range typical for that manufactured polymer, repeatedly softening when hot and, hardening when cold and, in the softened state, repeatedly being mouldable into intermediate or final articles by flowing, as a moulded, extruded or formed part for example. Advantageously, the covering layer is embodied as a semipermeable membrane, i.e. as a membrane which retains wound exudate passing through the foam layer and also water from the outside, but allows water vapour to pass.

It will further be found particularly advantageous when the membranes or foils have thicknesses in the range from ≧5 μm to ≦80 μm, in particular from ≧5 μm to ≦60 μm and more preferably from ≧10 μm to ≦30 μm and a breaking extension of above 450%.

In the present invention, the covering layer and the foam layer are at least partly bonded together directly. As a result, there are regions in this layered composite in which the two layers are in immediate superposition. In these regions, the layered composite is notable precisely for the fact that there is no adhesive between the foam layer and the covering layer.

The polyurethane dispersion (I) comprises polyurethanes prepared by reacting free isocyanate groups as a whole or in part with anionic or potentially anionic hydrophilicizing agents. Such hydrophilicizing agents are compounds which have isocyanate-reactive functional groups such as amino, hydroxyl or thiol groups as well as acid or acid anion groups such as carboxylate, sulphonate or phosphonate groups.

The foam layer and the covering layer are selected according to the present invention to obtain wound dressings comprising a protective, covering and/or membrane foil which are obtainable in fewer operations as a result of the need for an adhesive layer being eliminated. Yet interadhesion of the layers is sufficient. After production, the layered composites continue to be further processable by means of thermoforming, making it possible to achieve a large diversity of shapes for the wound dressings.

In one embodiment of the layered composite according to the invention, the composition from which the polyurethane foam of the foam layer is obtained further comprises admixtures selected from the group comprising fatty acid amides, sulphosuccinamides, hydrocarbonsulphonates, hydrocarbyl sulphates, fatty acid salts, alkylpolyglycosides and/or ethylene oxide-propylene oxide block copolymers.

Such admixtures can act as foam formers and/or foam stabilizers. The lipophilic radical in the fatty acid amides, sulphosuccinamides, hydrocarbonsulphonates, hydrocarbyl sulphates or fatty acid salts preferably comprises ≧12 to ≦24 carbon atoms. Suitable alkylpolyglycosides are obtainable for example by reaction of comparatively long-chain monoalcohols (≧4 to ≦22 carbon atoms in the alkyl radical) with mono-, di- or polysaccharides. Also suitable are alkylbenzosulphonates or alkylbenzene sulphates having ≧14 to ≦24 carbon atoms in the hydrocarbyl radical.

The fatty acid amides are preferably those based on mono- or di-(C₂/C₃-alkanol)amines. The fatty acid salts can be for example alkali metal salts, amine salts or unsubstituted ammonium salts.

Such fatty acid derivatives are typically based on fatty acids such as lauric acid, myristic acid, palmitic acid, oleic acid, stearic acid, ricinoleic acid, behenic acid or arachidic acid, coco fatty acid, tallow fatty acid, soya fatty acid and hydrogenation products thereof.

Exemplarily useful foam stabilizers are mixtures of sulphosuccinamides and ammonium stearates, the ammonium stearate content being preferably ≧20% by weight to ≦60% by weight, more preferably ≧30% by weight to ≦50% by weight, and the sulphosuccinamide content being preferably ≧40% by weight, to ≦80% by weight, more preferably ≧50% by weight to ≦70% by weight.

Further exemplarily useful foam stabilizers are mixtures of fatty alcohol-polyglycosides and ammonium stearates, the ammonium stearate content being preferably ≧20% by weight to ≦60% by weight and more preferably ≧30% by weight to ≦50% by weight and the fatty alcohol-polyglycoside content being preferably ≧40% by weight to ≦80% by weight and more preferably ≧50% by weight to ≦70% by weight.

The ethylene oxide-propylene oxide block copolymers comprise addition products of ethylene oxide and propylene oxide onto OH- or NH-functional starter molecules.

Useful starter molecules include in principle inter alia water, polyethylene glycols, polypropylene glycols, glycerol, trimethylolpropane, pentaerythritol, ethylenediamine, tolylenediamine, sorbitol, sucrose and mixtures thereof.

Preference is given to using di- or trifunctional compounds of the aforementioned kind as starters. Particular preference is given to polyethylene glycol or polypropylene glycol.

By varying the amount of alkylene oxide in each case and the number of ethylene oxide (EO) and propylene oxide (PO) blocks it is possible to obtain block copolymers of various kinds.

It is also possible in principle for copolymers constructed strictly blockwise from ethylene oxide or propylene oxide to also include individual mixed blocks of EO and PO.

Such mixed blocks are obtained on using mixtures of EO and PO in the polyaddition reaction so that, in relation to this block, a random distribution of EO and PO results in this block.

The ethylene oxide content of the EO/PO block copolymers used according to the invention is preferably ≧5% by weight, more preferably ≧20% by weight and most preferably ≧40% by weight, based on the sum total of the ethylene oxide and propylene oxide units present in the copolymer.

The ethylene oxide content of the EO/PO block copolymers used according to the invention is preferably ≦95% by weight, more preferably ≦90% by weight and most preferably ≦85% by weight based on the sum total of the ethylene oxide and propylene oxide units present in the copolymer.

The number average molecular weight of the EO/PO block copolymers used according to the invention is preferably ≧1000 g/mol, more preferably ≧2000 g/mol and most preferably ≧5000 g/mol.

The number average molecular weight of the EO/PO block copolymers used according to the invention is preferably ≦10 000 g/mol, more preferably ≦9500 g/mol and most preferably ≦9000 g/mol.

One advantageous aspect of using the EO/PO block copolymers is that the foam obtained has a lower hydrophobicity than when other stabilizers are used. The imbibition behaviour for liquids can be favourably influenced as a result. Moreover, non-cytotoxic foams are obtained when EO/PO block copolymers are used, in contradistinction to other stabilizers.

It is preferred for the ethylene oxide-propylene oxide block copolymers to have a structure conforming to the general formula (I):

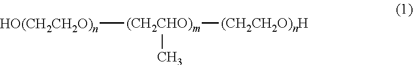

where n is in the range from ≧2 to ≦200, and m is in the range from ≧10 to ≦60.
n is preferably in the range from ≧60 to ≦180, more preferably from ≧130 to ≦160. m is preferably in the range from ≧25 to ≦45, more preferably from ≧25 to ≦35.

EO/PO block copolymers of the aforementioned kind are particularly preferred in that they have a hydrophilic-lipophilic balance (HLB) of ≧4, more preferably of ≧8 and most preferably of ≧14. The HLB value computes according to the formula HLB=20·Mh/M, where Mh is the number average molar mass of the hydrophilic moiety, formed from ethylene oxide, and M is the number average molar mass of the overall molecule (Griffin, W. C.: Classification of surface active agents by HLB, J. Soc. Cosmet. Chem. 1, 1949). However, the HLB value is ≦19 and preferably ≦18.

In one embodiment of the layered composite of the invention, the aqueous, anionically hydrophilicized polyurethane dispersion (I) is obtainable by
A) providing isocyanate-functional prepolymers obtainable from a reaction mixture comprising
   A1) organic polyisocyanates and
   A2) polymeric polyols having number average molecular weights of ≧400 g/mol to ≦8000 g/mol and OH functionalities of ≧1.5 to ≦6 and subsequently
B) reacting the free NCO groups of the prepolymers in whole or in part with
   B1) amino-functional anionic or potentially anionic hydrophilicizing agents
      with chain extension and dispersing the prepolymers in water before, during or after step B), wherein potentially ionic groups still present in the reaction mixture are converted into their ionic form by partial or complete reaction with a neutralizing agent.

Preferred aqueous, anionic polyurethane dispersions (I) have a low degree of hydrophilic anionic groups, preferably in the range from ≧0.1 to ≦15 milliequivalents per 100 g of solid resin.

To achieve good sedimentation stability, the number average particle size of the specific polyurethane dispersions is preferably ≦750 nm and more preferably ≦500 nm, determined by means of laser correlation spectroscopy.

The ratio of NCO groups of compounds of component A1) to NCO-reactive groups such as amino, hydroxyl or thiol groups of compounds of components A2) to A4) is ≧1.05 to ≦3.5, preferably ≧1.2 to ≦3.0 and more preferably ≧1.3 to ≦2.5 to prepare the NCO-functional prepolymer.

The amino-functional compounds in stage B) are used in such an amount that the equivalent ratio of isocyanate-reactive amino groups of these compounds to the free isocyanate groups of the prepolymer is ≧40% to ≦150%, preferably between ≧50% and ≦125% and more preferably between ≧60% and ≦120%.

Suitable polyisocyanates for component A1) are aromatic, araliphatic, aliphatic or cycloaliphatic polyisocyanates of an NCO functionality of ≧2.

Examples of such suitable polyisocyanates are 1,4-butylene diisocyanate, 1,6-hexamethylene diiso-cyanate (HDI), isophorone diisocyanate (IPDI), 2,2,4- and/or 2,4,4-trimethylhexamethylene diisocyanate, the isomeric bis(4,4′-isocyanatocyclohexyl)methanes or their mixtures of any desired isomer content, 1,4-cyclohexylene diisocyanate, 1,4-phenylene diisocyanate, 2,4- and/or 2,6-tolylene diisocyanate (TDI), 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI), and also alkyl 2,6-diisocyanatohexanoates (lysine diisocyanates) having C₁-C₈-alkyl groups.

As well as the aforementioned polyisocyanates, it is also possible to use, proportionally, modified diisocyanates of uretdione, isocyanurate, urethane, allophanate, biuret, iminooxadiazinedione and/or oxadiazinetrione structure and also non-modified polyisocyanate having more than 2 NCO groups per molecule, for example 4-isocyanatomethyl-1,8-octane diisocyanate (nonane triisocyanate) or triphenylmethane 4,4′,4″-triisocyanate.

Preferably, the polyisocyanates or polyisocyanate mixtures of the aforementioned kind have exclusively aliphatically and/or cycloaliphatically attached isocyanate groups and an average NCO functionality of ≧2 to ≦4, preferably ≧2 to ≦2.6 and more preferably ≧2 to ≦2.4 for the mixture.

It is particularly preferable for A1) to utilize 1,6-hexamethylene diisocyanate, isophorone diisocyanate, the isomeric bis(4,4′-isocyanatocyclohexyl)methanes, and also mixtures thereof.

A2) utilizes polymeric polyols having a number average molecular weight Mₙ of ≧400 g/mol to ≦8000 g/mol, preferably from ≧400 g/mol to ≦6000 g/mol and more preferably from ≧600 g/mol to ≦3000 g/mol. These preferably have an OH functionality of ≧1.5 to ≦6, more preferably of ≧1.8 to ≦3 and most preferably of ≧1.9 to ≦2.1.

Such polymeric polyols include for example polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols. These can be used in A2) individually or in any desired mixtures with one another.

Such polyester polyols include polycondensates formed from di- and also optionally tri- and tetraols and di- and also optionally tri- and tetracarboxylic acids or hydroxy carboxylic acids or lactones. Instead of the free polycarboxylic acids it is also possible to use the corresponding polycarboxylic anhydrides or corresponding polycarboxylic esters of lower alcohols for preparing the polyesters.

Examples of suitable diols are ethylene glycol, butylene glycol, diethylene glycol, triethylene glycol, polyalkylene glycols such as polyethylene glycol, also 1,2-propanediol, 1,3-propanediol, butanediol(1,3), butanediol(1,4), hexanediol(1,6) and isomers, neopentyl glycol or neopentyl glycol hydroxypivalate, of which hexanediol(1,6) and isomers, neopentyl glycol and neopentyl glycol hydroxypivalate are preferred. Besides these it is also possible to use polyols such as trimethylolpropane, glycerol, erythritol, pentaerythritol, trimethylolbenzene or trishydroxyethyl isocyanurate.

Useful dicarboxylic acids include phthalic acid, isophthalic acid, terephthalic acid, tetra-hydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethyl glutaric acid and/or 2,2-dimethylsuccinic acid. The corresponding anhydrides can also be used as a source of an acid.

When the average functionality of the polyol to be esterified is ≧2, monocarboxylic acids, such as benzoic acid and hexanecarboxylic acid can be used as well in addition.

Preferred acids are aliphatic or aromatic acids of the aforementioned kind. Adipic acid, isophthalic acid and optionally trimellitic acid are particularly preferred.

Hydroxy carboxylic acids useful as reaction participants in the preparation of a polyester polyol having terminal hydroxyl groups include for example hydroxycaproic acid, hydroxybutyric acid, hydroxydecanoic acid, hydroxystearic acid and the like. Suitable lactones include caprolactone, butyrolactone and homologues. Caprolactone is preferred.

A2) may likewise utilize hydroxyl-containing polycarbonates, preferably polycarbonate diols, having number average molecular weights Mₙ of ≧400 g/mol to ≦8000 g/mol and preferably in the range from ≧600 g/mol to ≦3000 g/mol. These are obtainable by reaction of carbonic acid derivatives, such as diphenyl carbonate, dimethyl carbonate or phosgene, with polyols, preferably diols.

Examples of such diols are ethylene glycol, 1,2-propanediol, 1,3-propanediol, 1,3-butanediol, 1,4-butanediol, 1,6-hexanediol, 1,8-octanediol, neopentyl glycol, 1,4-bishydroxymethyl-cyclohexane, 2-methyl-1,3-propanediol, 2,2,4-trimethyl-1,3-pentanediol, dipropylene glycol, polypropylene glycols, dibutylene glycol, polybutylene glycols, bisphenol A and lactone-modified diols of the aforementioned kind.

The polycarbonate diol preferably contains ≧40% to ≦100% by weight of hexanediol, preference being given to 1,6-hexanediol and/or hexanediol derivatives. Such hexanediol derivatives are based on hexanediol and have ester or ether groups as well as terminal OH groups. Such derivatives are obtainable by reaction of hexanediol with excess caprolactone or by etherification of hexanediol with itself to form di- or trihexylene glycol.

In lieu of or in addition to pure polycarbonate diols, polyether-polycarbonate diols can also be used in A2).

Hydroxyl-containing polycarbonates preferably have a linear construction.

A2) may likewise utilize polyether polyols.

Useful for example are polytetramethylene glycol polyethers as are obtainable by polymerization of tetrahydrofuran by means of cationic ring opening.

Useful polyether polyols likewise include the addition products of styrene oxide, ethylene oxide, propylene oxide, butylene oxide and/or epichlorohydrin onto di- or polyfunctional starter molecules. Polyether polyols based on the at least proportional addition of ethylene oxide onto di- or polyfunctional starter molecules can also be used as component A4) (nonionic hydrophilicizing agents).

Useful starter molecules include for example water, butyl diglycol, glycerol, diethylene glycol, trimethylolpropane, propylene glycol, sorbitol, ethylenediamine, triethanolamine, or 1,4-butanediol. Preferred starter molecules are water, ethylene glycol, propylene glycol, 1,4-butanediol, diethylene glycol and butyl diglycol.

Particularly preferred embodiments of the polyurethane dispersions (I) contain as component A2) a mixture of polycarbonate polyols and polytetramethylene glycol polyols, the proportion of polycarbonate polyols in this mixture being ≧20% to ≦80% by weight and the proportion of polytetramethylene glycol polyols in this mixture being ≧20% to ≦80% by weight. Preference is given to a proportion of ≧30% to ≦75% by weight for polytetramethylene glycol polyols and to a proportion of ≧25% to ≦70% by weight for polycarbonate polyols. Particular preference is given to a proportion of ≧35% to ≦70% by weight for polytetramethylene glycol polyols and to a proportion of ≧30% to ≦65% by weight for polycarbonate polyols, each subject to the proviso that the sum total of the weight percentages for the polycarbonate and polytetramethylene glycol polyols is ≦100% by weight and the proportion of component A2) which is accounted for by the sum total of the polycarbonate polyols and polytetramethylene glycol polyether polyols is ≧50% by weight, preferably ≧60% by weight and more preferably ≧70% by weight.

An isocyanate-reactive anionic or potentially anionic hydrophilicizing agent of component B1) is any compound which has at least one isocyanate-reactive group such as an amino, hydroxyl or thiol group and also at least one functionality such as for example —COO⁻M⁺, —SO₃⁻M⁺, —PO(O⁻M⁺)₂ where M⁺ is for example a metal cation, H⁺, NH₄⁺, NHR₃⁺, where R in each occurrence may be C₁-C₁₂-alkyl, C₅-C₆-cycloalkyl and/or C₂-C₄-hydroxyalkyl, which functionality on interaction with aqueous media enters a pH-dependent dissociative equilibrium and thereby can have a negative or neutral charge.

The isocyanate-reactive anionic or potentially anionic hydrophilicizing agents are preferably isocyanate-reactive amino-functional anionic or potentially anionic hydrophilicizing agents.

Useful anionically or potentially anionically hydrophilicizing compounds are mono- and diamino carboxylic acids, mono- and diamino sulphonic acids and also mono- and diamino phosphonic acids and their salts. Examples of such anionic or potentially anionic hydrophilicizing agents are N-(2-aminoethyl)-β-alanine, 2-(2-aminoethylamino)ethanesulphonic acid, ethylenediaminepropyl-sulphonic acid, ethylenediaminebutylsulphonic acid, 1,2- or 1,3-propylenediamine-β-ethyl-sulphonic acid, glycine, alanine, taurine, lysine, 3,5-diaminobenzoic acid and the addition product of IPDA and acrylic acid (EP-A 0 916 647, Example 1). It is further possible to use cyclohexyl-aminopropanesulphonic acid (CAPS) from WO-A 01/88006 as anionic or potentially anionic hydrophilicizing agent.

Preferred anionic or potentially anionic hydrophilicizing agents for component B1) are those of the aforementioned kind that have carboxylate or carboxyl groups and/or sulphonate groups, such as the salts of N-(2-aminoethyl)-β-alanine, of 2-(2-aminoethylamino)ethanesulphonic acid or of the addition product of IPDA and acrylic acid (EP-A 0 916 647, Example 1).

Mixtures of anionic or potentially anionic hydrophilicizing agents and nonionic hydrophilicizing agents can also be used.

In a further embodiment of the layered composite of the present invention, the reaction mixture in step A) further comprises:
A3) hydroxyl-functional compounds having molecular weights of ≧62 g/mol to ≦399 g/mol.

The compounds of component A3) have molecular weights of ≧62 to ≦399 g/mol.

A3) may utilize polyols of the specified molecular weight range with up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxy-phenyl)propane), hydrogenated bisphenol A, (2,2-bis(4-hydroxycyclohexyl)propane), trimethylol-propane, glycerol, pentaerythritol and also any desired mixtures thereof with one another.

Also suitable are ester diols of the specified molecular weight range such as α-hydroxybutyl-ε-hydroxycaproic acid ester, ω-hydroxyhexyl-γ-hydroxybutyric acid ester, β-hydroxyethyl adipate or bis(β-hydroxyethyl)terephthalate.

A3) may further utilize monofunctional isocyanate-reactive hydroxyl-containing compounds. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Preferred compounds for component A3) are 1,6-hexanediol, 1,4-butanediol, neopentyl glycol and trimethylolpropane.

In a further embodiment of the layered composite of the invention, the reaction mixture in step A) further comprises:
A4) isocyanate-reactive anionic or potentially anionic and optionally nonionic hydrophilicizing agents.

An anionically or potentially anionically hydrophilicizing compound for component A4) is any compound which has at least one isocyanate-reactive group such as an amino, hydroxyl or thiol group and also at least one functionality such as for example —COO⁻M⁺, —SO₃⁻M⁺, —PO(O⁻M⁺)₂ where M⁺ is for example a metal cation, H⁺, NH₄⁺, NHR₃⁺, where R in each occurrence may be C₁-C₁₂-alkyl, C₅-C₆-cycloalkyl and/or C₂-C₄-hydroxyalkyl, which functionality enters on interaction with aqueous media a pH-dependent dissociative equilibrium and thereby can have a negative or neutral charge. Useful anionically or potentially anionically hydrophilicizing compounds include for example mono- and dihydroxy carboxylic acids, mono- and dihydroxy sulphonic acids and also mono- and dihydroxy phosphonic acids and their salts. Examples of such anionic or potentially anionic hydrophilicizing agents are dimethylolpropionic acid, dimethylolbutyric acid, hydroxypivalic acid, malic acid, citric acid, glycolic acid, lactic acid and the propoxylated adduct formed from 2-butenediol and NaHSO₃ as described in DE-A 2 446 440, page 5-9, formula I-III. Preferred anionic or potentially anionic hydrophilicizing agents for component A4) are those of the aforementioned kind that have carboxylate or carboxyl groups and/or sulphonate groups.

Particularly preferred anionic or potentially anionic hydrophilicizing agents are those that contain carboxylate or carboxyl groups as ionic or potentially ionic groups, such as dimethylolpropionic acid, dimethylolbutyric acid and hydroxypivalic acid and/or salts thereof.

Useful nonionically hydrophilicizing compounds for component A4) include for example polyoxyalkylene ethers which contain at least one hydroxyl or amino group, preferably at least one hydroxyl group. Examples thereof are the monohydroxyl-functional polyalkylene oxide polyether alcohols containing on average ≧5 to ≦70 and preferably ≧7 to ≦55 ethylene oxide units per molecule and obtainable by alkoxylation of suitable starter molecules. These are either pure polyethylene oxide ethers or mixed polyalkylene oxide ethers, containing ≧30 mol % and preferably ≧40 mol % of ethylene oxide units, based on all alkylene oxide units present.

Preferred polyethylene oxide ethers of the aforementioned kind are monofunctional mixed polyalkylene oxide polyethers having ≧40 mol % to ≦100 mol % of ethylene oxide units and ≧0 mol % to ≦60 mol % of propylene oxide units.

Preferred nonionically hydrophilicizing compounds for component A4) include those of the aforementioned kind that are block (co)polymers prepared by blockwise addition of alkylene oxides onto suitable starters.

Useful starter molecules for such nonionic hydrophilicizing agents include saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or oleic alcohol, aromatic alcohols such as phenol, the isomeric cresols or methoxyphenols, araliphatic alcohols such as benzyl alcohol, anis alcohol or cinnamyl alcohol, secondary monoamines such as dimethylamine, diethylamine, dipropylamine, diisopropylamine, dibutylamine, bis(2-ethylhexyl)amine, N-methylcyclo-hexylamine, N-ethylcyclohexylamine or dicyclohexylamine and also heterocyclic secondary amines such as morpholine, pyrrolidine, piperidine or 1H pyrazole. Preferred starter molecules are saturated monoalcohols of the aforementioned kind. Particular preference is given to using diethylene glycol monobutyl ether or n-butanol as starter molecules.

Useful alkylene oxides for the alkoxylation reaction are in particular ethylene oxide and propylene oxide, which can be used in any desired order or else in admixture in the alkoxylation reaction.

In a further embodiment of the layered composite of the invention, the free NCO groups of the prepolymers are further reacted in whole or in part in step B) with
B2) amino-functional compounds having molecular weights of ≧32 g/mol to ≦400 g/mol.

Component B2) may utilize di- or polyamines such as 1,2-ethylenediamine, 1,2-diaminopropane, 1,3-diaminopropane, 1,4-diaminobutane, 1,6-diaminohexane, isophoronediamine, isomeric mixtures of 2,2,4- and 2,4,4-trimethylhexamethylenediamine, 2-methylpentamethylenediamine, diethylenetriamine, triaminononane, 1,3-xylylenediamine, 1,4-xylylenediamine, α,α,α′,α′-tetramethyl-1,3- and -1,4-xylylenediamine and 4,4-diaminodicyclohexylmethane and/or dimethylethylenediamine. It is also possible but less preferable to use hydrazine and also hydrazides such as adipohydrazide.

Component B2) can further utilize compounds which as well as a primary amino group also have secondary amino groups or which as well as an amino group (primary or secondary) also have OH groups. Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

Component B2) can further utilize monofunctional isocyanate-reactive amine compounds, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide-amines formed from diprimary amines and monocarboxylic acids, monoketimes of diprimary amines, primary/tertiary amines, such as N,N-dimethylaminopropylamine. Preferred compounds for component B2) are 1,2-ethylenediamine, 1,4-diaminobutane and isophoronediamine.

In a further embodiment of the layered composite of the invention, in the preparation of the aqueous, anionically hydrophilicized polyurethane dispersions (I), the component A1) is selected from the group comprising 1,6-hexamethylene diisocyanate, isophorone diisocyanate and/or the isomeric bis(4,4′-isocyanatocyclohexyl)methanes. The component A2) furthermore comprises a mixture of polycarbonate polyols and polytetramethylene glycol polyols, wherein the proportion of component A2) which is accounted for by the sum total of the polycarbonate polyols and the polytetramethylene glycol polyether polyols is ≧70% by weight to ≦100% by weight.

In addition to the polyurethane dispersions (I) and the admixtures, it is also possible to use further auxiliary materials.

Examples of such auxiliary materials are thickeners/thixotropic agents, antioxidants, photostabilizers, emulsifiers, plasticizers, pigments, fillers and/or flow control agents.

Commercially available thickeners can be used, such as derivatives of dextrin, of starch or of cellulose, examples being cellulose ethers or hydroxyethylcellulose, polysaccharide derivatives such as gum arabic or guar, organic wholly synthetic thickeners based on polyacrylic acids, polyvinylpyrrolidones, poly(meth)acrylic compounds or polyurethanes (associative thickeners) and also inorganic thickeners such as bentonites or silicas.

In principle, the compositions of the invention can also contain crosslinkers such as unblocked polyisocyanates, amide- and amine-formaldehyde resins, phenolic resins, aldehydic and ketonic resins, examples being phenol-formaldehyde resins, resols, furan resins, urea resins, carbamic ester resins, triazine resins, melamine resins, benzoguanamine resins, cyanamide resins or aniline resins.

In a further embodiment of the layered composite of the present invention, the thermoplastic polymer of the covering layer comprises materials selected from the group comprising polyurethane, polyethylene, polypropylene, polyvinyl chloride, polystyrene, polyether, polyester, polyamide, polycarbonate, polyether-polyamide copolymers, polyacrylate, polymethacrylate and/or polymaleate. The material in question preferably comprises thermoplastic polyurethane (TPU). Advantageously, these materials are elastomeric. Also favourable here are foils of such materials in a thickness of ≧5 μm to ≦80 μm, in particular ≧5 μm to ≦60 μm and more preferably ≧10 μm to ≦30 μm.

In a further embodiment of the layered composite of the present invention, the thermoplastic polymer of the covering layer comprises polyurethanes selected from the group comprising aliphatic polyester polyurethanes, aromatic polyester polyurethanes, aliphatic polyether polyurethanes and/or aromatic polyether polyurethanes. It is thus possible to obtain breathable elastic membrane foils. The covering layers thus obtainable are notable for high flexibility/elasticity over a wide temperature range, good wind and water impermeability combined with high water vapour permeability, low noise, good textile haptics, durability to washing and cleaning, very good chemical and mechanical durability and absence of plasticizer.

In a further embodiment of the layered composite of the present invention, the direct bond between the foam layer and the covering layer has a peel strength of ≧0.01 N/mm to ≦0.50 N/mm. Peel strength can also be in a range from ≧0.03 N/mm to ≦0.30 N/mm or from ≧0.05 N/mm to ≦0.20 N/mm. Peel strength can be determined by performing 360° peel tests on a Zwick Universal Tester at a traverse speed of 100 mm/min.

In a further embodiment of the layered composite of the present invention, the water vapour permeability of the layered composite is in the range from ≧1000 g/24 h×m² to ≦4000 g/24 h×m². This water vapour permeability can also be in the range from ≧1500 g/24 h×m² to ≦3000 g/24 h×m² or from ≧1800 g/24 h×m² to ≦2500 g/24 h×m².

It is possible for the covering layer in the layered composite of the present invention to have an impermeability to water, expressed as water column on the layer, of ≧2000 mm. This value can also be in the range of ≧4000 mm or ≧6000 mm.

Furthermore, the covering layer can have a water vapour permeability of ≧1000 g/24 h×m² to ≦8000 g/24 h×m². This water vapour permeability can also be in the range from ≧2000 g/24 h×m² to ≦6000 g/24 h×m² or from ≧3000 g/24 h×m² to ≦5000 g/24 h×m².

An exemplary recipe for preparing the polyurethane dispersions utilizes the components A1) to A4) and B1) to B2) in the following amounts, the individual amounts always adding up to ≦100% by weight:

≧5% by weight to ≦40% by weight of component A1);
≧55% by weight to ≦90% by weight of component A2);
≧0.5% by weight to ≦20% by weight of the sum total of components A3) and B2);
≧0.1% by weight to ≦25% by weight of the sum total of components A4) and B1), wherein, based on the total amounts of the components A1) to A4) and B1) to B2), ≧0.1% by weight to ≦5% by weight of anionic or potentially anionic hydrophilicizing agents from A4) and/or B1) are used.

A further exemplary recipe for preparing the polyurethane dispersions utilizes the components A1) to A4) and B1) to B2) in the following amounts, the individual amounts always adding up to ≦100% by weight:

≧5% by weight to ≦35% by weight of component A1);
≧60% by weight to ≦90% by weight of component A2);
≧0.5% by weight to ≦15% by weight of the sum total of components A3) and B2);
≧0.1% by weight to ≦15% by weight of the sum total of components A4) and B1), wherein, based on the total amounts of the components A1) to A4) and B1) to B2), 0.2% by weight to ≦4% by weight of anionic or potentially anionic hydrophilicizing agents from A4) and/or B1) are used.

A very particularly preferred recipe for preparing the polyurethane dispersions utilizes the components A1) to A4) and B1) to B2) in the following amounts, the individual amounts always adding up to ≦100% by weight:

≧10% by weight to ≦30% by weight of component A1);
≧65% by weight to ≦85% by weight of component A2);
≧0.5% by weight to ≦14% by weight of the sum total of components A3) and B2);
≧0.1% by weight to ≦13.5% by weight of the sum total of components A4) and B1), wherein, based on the total amounts of the components A1) to A4) and B1) to B2), 0.5% by weight to ≦3.0% by weight of anionic or potentially anionic hydrophilicizing agents from A4) and/or B1) are used.

The production of the anionically hydrophilicized polyurethane dispersions (I) can be carried out in one or more stages in homogeneous phase or, in the case of a multistage reaction, partly in disperse phase. After completely or partially conducted polyaddition from A1) to A4), a dispersing, emulsifying or dissolving step is carried out. This is followed if appropriate by a further polyaddition or modification in disperse phase.

Processes such as for example the prepolymer mixing process, the acetone process or the melt dispersing process can be used. The acetone process is preferred.

Production by the acetone process typically involves the constituents A2) to A4) and the polyisocyanate component A1) being wholly or partly introduced as an initial charge to produce an isocyanate-functional polyurethane prepolymer and optionally diluted with a water-miscible but isocyanate-inert solvent and heated to temperatures in the range from ≧50 to ≦120° C. The isocyanate addition reaction can be speeded using the catalysts known in polyurethane chemistry.

Useful solvents include the customary aliphatic, keto-functional solvents such as acetone or 2-butanone, which can be added not just at the start of the production process but also later, optionally in portions. Acetone and 2-butanone are preferred.

Other solvents such as xylene, toluene, cyclohexane, butyl acetate, methoxypropyl acetate, N-methylpyrrolidone, N-ethylpyrrolidone, solvents having ether or ester units can additionally be used and wholly or partly distilled off or in the case of N-methylpyrrolidone, N-ethylpyrrolidone remain completely in the dispersion. But preference is given to not using any other solvents apart from the customary aliphatic, keto-functional solvents.

Subsequently, any constituents of A1) to A4) not added at the start of the reaction are added.

In the production of the polyurethane prepolymer from A1) to A4), the amount of substance ratio of isocyanate groups to with isocyanate-reactive groups is for example in the range from ≧1.05 to ≦3.5, preferably in the range from ≧1.2 to ≦3.0 and more preferably in the range from ≧1.3 to ≦2.5.

The reaction of components A1) to A4) to form the prepolymer is effected partially or completely, but preferably completely. Polyurethane prepolymers containing free isocyanate groups are obtained in this way, without a solvent or in solution.

The neutralizing step to effect partial or complete conversion of potentially anionic groups into anionic groups utilizes bases such as tertiary amines, for example trialkylamines having ≧1 to ≦12 and preferably ≧1 to ≦6 carbon atoms and more preferably ≧2 to ≦3 carbon atoms in every alkyl radical or alkali metal bases such as the corresponding hydroxides.

Examples thereof are trimethylamine, triethylamine, methyldiethylamine, tripropylamine, N-methylmorpholine, methyldiisopropylamine, ethyldiisopropylamine and diisopropylethylamine. The alkyl radicals may also bear for example hydroxyl groups, as in the case of the dialkylmonoalkanol-, alkyldialkanol- and trialkanolamines. Useful neutralizing agents further include if appropriate inorganic bases, such as aqueous ammonia solution, sodium hydroxide or potassium hydroxide.

Preference is given to ammonia, triethylamine, triethanolamine, dimethylethanolamine or diisopropylethylamine and also sodium hydroxide and potassium hydroxide, particular preference being given to sodium hydroxide and potassium hydroxide.

The bases are employed in an amount of substance which is between ≧50 and ≦125 mol % and preferably between ≧70 and ≦100 mol % of the amount of substance of the acid groups to be neutralized. Neutralization can also be effected at the same time as the dispersing step, by including the neutralizing agent in the water of dispersion.

Subsequently, in a further process step, if this has not already been done or only to some extent, the prepolymer obtained is dissolved with the aid of aliphatic ketones such as acetone or 2-butanone.

In the chain extension of stage B), NH₂- and/or NH-functional components are reacted, partially or completely, with the still remaining isocyanate groups of the prepolymer. Preferably, the chain extension is carried out before dispersion in water.

Chain termination is typically carried out using amines B2) having an isocyanate-reactive group such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine or suitable substituted derivatives thereof, amide-amines formed from diprimary amines and monocarboxylic acids, monoketimes of diprimary amines, primary/tertiary amines, such as N,N-dimethylaminopropylamine.

When partial or complete chain extension is carried out using anionic or potentially anionic hydrophilicizing agents conforming to definition B1) with NH₂ or NH groups, chain extension of the prepolymers is preferably carried out before dispersion.

The aminic components B1) and B2) can optionally be used in water- or solvent-diluted form in the process of the invention, individually or in mixtures, any order of addition being possible in principle.

When water or organic solvent is used as a diluent, the diluent content of the chain-extending component used in B) is preferably in the range from ≧70% to ≦95% by weight.

Dispersion is preferably carried out following chain extension. For dispersion, the dissolved and chain-extended polyurethane polymer is either introduced into the dispersing water, if appropriate by substantial shearing, such as vigorous stirring for example, or conversely the dispersing water is stirred into the chain-extended polyurethane polymer solutions. It is preferable to add the water to the dissolved chain-extended polyurethane polymer.

The solvent still present in the dispersions after the dispersing step is then typically removed by distillation. Removal during the dispersing step is likewise possible.

The residual level of organic solvents in the polyurethane dispersions (I) is typically ≦1.0% by weight and preferably ≦0.5% by weight, based on the entire dispersion.

The pH of the polyurethane dispersions (I) of the present invention is typically ≦9.0, preferably ≦8.5, more preferably ≦8.0 and most preferably is in the range from ≧6.0 to ≦7.5.

The solids content of the polyurethane dispersions (I) is preferably in the range from ≧40% to ≦70% by weight, more preferably in the range from ≧50% to ≦65% by weight, even more preferably in the range from ≧55% to ≦65% by weight and in particular in the range from ≧60% to ≦65% by weight.

Examples of compositions according to the invention are recited hereinbelow, the sum total of the weights in % ages having a value of ≦100% by weight. These compositions, based on dry substance, typically comprise ≧80 parts by weight to ≦99.5 parts by weight of dispersion (I), ≧0 parts by weight to ≦10 parts by weight of foam auxiliary, ≧0 parts by weight to ≦10 parts by weight of crosslinker and ≧0 parts by weight to ≦10 parts by weight of thickener.

These compositions according to the invention, based on dry substance, preferably comprise ≧85 parts by weight to ≦97 parts by weight of dispersion (I), ≧0.5 part by weight to ≦7 parts by weight of foam auxiliary, ≧0 parts by weight to ≦5 parts by weight of crosslinker and ≧0 parts by weight to ≦5 parts by weight of thickener.

These compositions according to the invention, based on dry substance, more preferably comprise ≧89 parts by weight to ≦97 parts by weight of dispersion (I), ≧0.5 part by weight to ≦6 parts by weight of foam auxiliary, ≧0 parts by weight to ≦4 parts by weight of crosslinker and ≧0 parts by weight to ≦4 parts by weight of thickener.

Examples of compositions according to the invention which comprise ethylene oxide-propylene oxide block copolymers as foam stabilizers are recited hereinbelow. These compositions, based on dry substance, comprise ≧80 parts by weight to ≦99.9 parts by weight of dispersion (I) and ≧0.1 part by weight to ≦20 parts by weight of the ethylene oxide-propylene oxide block copolymers. The compositions, based on dry substance, preferably comprise ≧85 parts by weight to ≦99.5 parts by weight of dispersion (I) and 0.5 to 15 parts by weight of the ethylene oxide-propylene oxide block copolymers. Particular preference here is given to ≧90 parts by weight to ≦99 parts by weight of dispersion (I) and ≧1 part by weight to ≦10 parts by weight of the ethylene oxide-propylene oxide block copolymers and very particular preference is given to ≧94 parts by weight to ≦99 parts by weight of dispersion (I) and ≧1 to ≦6 parts by weight of the ethylene oxide-propylene oxide block copolymers.

For the purposes of the present invention, “parts by weight” denotes a relative proportion, but not in the sense of % by weight. Consequently, the arithmetic sum total of the proportions by weight can also assume values above 100.

In addition to the components mentioned, the compositions according to the invention may also utilize further aqueous binders. Such aqueous binders can be constructed for example of polyester, polyacrylate, polyepoxy or other polyurethane polymers. Similarly, the combination with radiation-curable binders as described for example in EP-A-0 753 531 is also possible. It is further possible to employ other anionic or nonionic dispersions, such as polyvinyl acetate, polyethylene, polystyrene, polybutadiene, polyvinyl chloride, polyacrylate and copolymer dispersions.

Frothing in the process of the present invention is accomplished by mechanical stirring of the composition at high speeds of rotation by shaking or by decompressing a blowing gas.

Mechanical frothing can be effected using any desired mechanical stirring, mixing and dispersing techniques. Air is generally introduced, but nitrogen and other gases can also be used for this purpose.

The invention further provides a process for producing a layered composite according to the present invention, comprising the steps of
providing a foam layer comprising a polyurethane foam obtained by a composition comprising an aqueous, anionically hydrophilicized polyurethane dispersion (I) being frothed;
applying a covering layer atop the foam layer.

The foam obtained is, in the course of frothing or immediately thereafter, applied atop a substrate or introduced into a mould and dried. Useful substrates include in particular papers, foils, films or sheets, which permit simple peeling off of the wound dressing before its use for covering an injured site.

Application can be for example by pouring or blade coating, but other conventional techniques are also possible. Multilayered application with intervening drying steps is also possible in principle. This can be followed by the application of the covering layer, followed by drying of the layered composite.

Alternatively, the foam layer may be directly blade coated atop the covering layer and the still moist layered composite dried.

A further alternative possibility is for the previously dried foam layer to have the covering layer laid on top, or for the covering layer to have the previously dried foam layer laid on top, and the layered composite obtained to be additionally heat conditioned.

Advantageously, however, the applying of the covering layer atop the foam layer is executed by laminating the covering layer atop the previously dried foam layer. Calendering machines for example can be used for laminating the covering layer atop the previously dried foam layer. The covering layer is advantageously applied under slight pressure in order that the adherence of the covering layer to the foam may be improved.

In general, a satisfactory drying rate for the foams is observed at a temperature as low as 20° C., so that drying on injured human or animal tissue presents no problem. However, temperatures above 30° C. are preferably used for more rapid drying and fixing of the foams. Temperatures of ≧80° C. to ≦160° C., preferably from ≧100° C. to ≦150° C. are more preferably from ≧120° C. to ≦140° C. are favourable. However, drying temperatures should not exceed 200° C., since undesirable yellowing of the foams can otherwise occur. Drying in two or more stages is also possible.

Drying is generally effected using conventional heating and drying apparatus, such as (circulating air) drying cabinets, hot air or IR radiators. Drying by leading the coated substrate over heated surfaces, for example rolls, is also possible.

Application and drying can each be carried out batchwise or continuously, but an entirely continuous process is preferred.

Before drying, the foam densities of the polyurethane foams are typically in a range from ≧50 g/litre to ≦800 g/litre, preferably ≧100 g/litre to ≦500 g/litre and more preferably ≧100 g/litre to ≦350 g/litre (mass of all input materials [in g] based on the foam volume of one litre).

After drying, the polyurethane foams can have a microporous, at least partly open-pore structure having intercommunicating cells. The density of the dried foams is typically below 0.4 g/cm³, preferably below 0.35 g/cm³, more preferably in the range from ≧0.01 g/cm³ to ≦0.3 g/cm³ and most preferably in the range from ≧0.1 g/cm³ to ≦0.3 g/cm³.

After drying, the thickness of the polyurethane foam layers, i.e. the foam layer and/or the covering layer, is typically in the range from ≧0.1 mm to ≦50 mm, preferably ≧0.5 mm to ≦20 mm, more preferably ≧1 mm to ≦10 mm and most preferably ≧1.5 mm to ≦5 mm.

The layered composite obtained can finally further be shaped under pressure and heat in order that it may be conformed to its later use.

The present invention further provides for the use of a layered composite according to the present invention as sports article, textile article, cosmetic article or wound dressing. The use as wound dressing is preferred. Advantageously, the wound dressing has such a shape that it can be laid onto body parts. An example of a body part is the heel, the forehead, the chin, the neck, the iliac crest or the buttock(s). The body part can further be a joint for example. With regard to its size, the wound dressing is conformed to the receiving body part such as the heel or a joint, i.e. for example a finger joint, an elbow joint, a knee joint or an ankle.

The present invention is further elucidated with reference to the following drawings, where

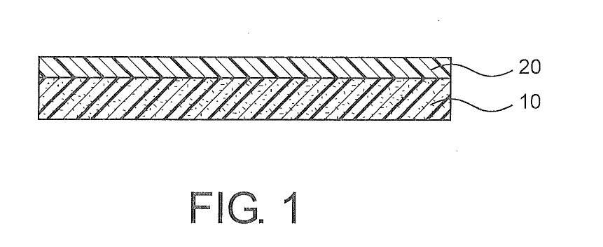

FIG. 1 shows a cross-sectional view of an inventive layered composite

FIG. 1 shows a cross-sectional view of an inventive layered composite. The foam layer 10 is embodied as a polyurethane foam layer, the polyurethane foam layer being obtainable as described. The covering layer 20, which is embodied as a thermoplastic polyurethane membrane, is in direct contact with the foam layer 10. On use of the layered composite, it is the lower surface of the foam layer 10 which is placed on the wound. The porous foam of the foam layer 10 is capable of absorbing surplus exudate from the wound. Water vapour can pass through the covering layer 20 and hence a climate beneficial to wound healing can be established in the wound. At the same time, the covering layer 20 protects the inferior foam layer 10 from getting dirty and colonized by microbes. The layered composite can subsequently be additionally shaped to obtain a wound dressing conformed to a body contour.

The present invention is further elucidated with reference to the examples which follow.

Unless indicated otherwise, all percentages are by weight.

Solids contents were determined in accordance with DIN-EN ISO 3251. NCO contents were, unless expressly mentioned otherwise, determined volumetrically in accordance with DIN-EN ISO 11909. “Free absorbency” was determined by absorption of physiological saline in accordance with DIN EN 13726-1 Part 3.2.

Substances and abbreviations used:
diaminosulphonate: NH₂—CH₂CH₂—NH—CH₂CH₂—SO₃Na (45% in water)
Desmophen® C2200: polycarbonate polyol, OH number 56 mg KOH/g, number average molecular weight 2000 g/mol (Bayer MaterialScience AG, Leverkusen, Germany)
PolyTHF® 2000: polytetramethylene glycol polyol, OH number 56 mg KOH/g, number average molecular weight 2000 g/mol (BASF AG, Ludwigshafen, Germany)
PolyTHF® 1000: polytetramethylene glycol polyol, OH number 112 mg KOH/g, number average molecular weight 1000 g/mol (BASF AG, Ludwigshafen, Germany)
LB 25 polyether: monofunctional polyether based on ethylene oxide-propylene oxide, number average molecular weight 2250 g/mol, OH number 25 mg KOH/g (Bayer MaterialScience AQ Leverkusen, Germany)
Pluronic® PE 6800: EO/PO block copolymer, weight average molecular weight 8000 g/mol (BASF AQ Ludwigshafen, Germany)
Foil 1: polyether-polyurethane membrane foil, water vapour permeability (DIN 53122; 38° C./90% rel. humidity): 4500 g/(m²×d)
Foil 2: polyether-polyurethane membrane foil, water vapour permeability (DIN 53122; 38° C./90% rel. humidity): 3000 g/(m²×d)
Foil 3: polyether-polyurethane membrane foil, water vapour permeability (DIN 53122; 38° C./90% rel. humidity): 2000 g/(m²×d)

The determination of the average particle sizes (the number average is reported) of polyurethane dispersion 1 was carried out using laser correlation spectroscopy (LCS; instrument: Malvern Zetasizer 1000, Malver Inst. Limited).

The contents reported for the foam additives are based on aqueous solutions.

### EXAMPLE 1

### Production of Polyurethane Dispersion 1

1077.2 g of PolyTHF® 2000, 409.7 g of PolyTHF® 1000, 830.9 g of Desmophen® C2200 and 48.3 g of LB 25 polyether were heated to 70° C. in a standard stirring apparatus. Then, a mixture of 258.7 g of hexamethylene diisocyanate and 341.9 g of isophorone diisocyanate was added at 70° C. in the course of 5 min and the mixture was stirred at 120° C. until the theoretical NCO value was reached or the actual NCO value was slightly below the theoretical NCO value. The ready-produced prepolymer was dissolved with 4840 g of acetone and, in the process, cooled down to 50° C. and subsequently admixed with a solution of 27.4 g of ethylenediamine, 127.1 g of isophoronediamine, 67.3 g of diaminosulphonate and 1200 g of water metered in over 10 min. The mixture was subsequently stirred for 10 min. Then, a dispersion was formed by addition of 654 g of water. This was followed by removal of the solvent by distillation under reduced pressure.

The polyurethane dispersion obtained had the following properties:

| | |
|---|---|
| Solids content: | 61.6% |
| Particle size (LCS): | 528 nm |
| pH (23° C.): | 7.5 |

### EXAMPLES 2-6

### Production of Foil-Foam Laminates from Polyurethane Dispersion 1

120 g of polyurethane dispersion 1, produced according to Example 1, were mixed with 12.6 g of a 30% solution of Pluronic® PE 6800 in water and frothed by means of a commercially available hand stirrer (stirrer made from bent wire) to a 0.4 litre foam volume. The foam was drawn down on non-adhesive paper by means of a blade coater set to a gap height of 6 mm and dried for 20 minutes at 120° C. in a circulating air drying cabinet. Subsequently, a foil of thermoplastic polyurethane (TPU foil; see table) was laid onto the dried foam and, by slight vaulting of the 2-layered material, fixed on the convex side of the foam without creases and bubbles. Then, the composite material was heat conditioned for 30 minutes in a drying cabinet at the temperature reported in the table. Clean white foil-foam laminates having good mechanical properties and a fine porous structure were obtained.

| | | Foil | Heat | MVTR | TFHC | Peel |
|---|---|---|---|---|---|---|
| | TPU | thickness | conditioning | [g/24 | [g/24 | strength |
| # | foil | [μm] | [° C.] | h/m²] | h/10 cm²] | [N/mm] |
| 2 | Foil 1 | 15 | 120 | 1840 | 15.9 | 0.03 |
| 3 | Foil 1 | 15 | 140 | 1650 | n.d. | 0.11 |
| 4 | Foil 2 | 15 | 120 | 1575 | 15.0 | n.d. |
| 5 | Foil 3 | 25 | 120 | 1137 | 9.3 | n.d. |
| 6 | Foil 3 | 30 | 120 | 1037 | 9.1 | n.d. |

n.d.: value not determined.
MVTR: moisture vapour transmission rate of wound dressings to DIN EN 13726-2 Part 3.2.
TFHC: total fluid handling capacity of a wound dressing on contact with fluid to DIN EN 13726-1 Part 3.3.
Peel strength: determined by performing 360° peel tests on a Zwick Universal Tester at a traverse speed of 100 mm/min.

The Examples show that the layered composites which are useful as wound dressing by virtue of their moisture vapour transmission rate and their moisture absorbency can be laminated with membrane foils.

1-15. (canceled)

16. A layered composite comprising a foam layer and a cover layer, wherein the cover layer comprises a thermoplastic polymer and is at least partly bonded directly to the foam layer, and wherein the foam layer comprises a polyurethane foam obtained by frothing and drying a composition comprising an aqueous, anionically hydrophilicized polyurethane dispersion.

17. The layered composite according to claim 16, wherein the composition from which the polyurethane foam is obtained further comprises components selected from the group consisting of fatty acid amides, sulphosuccinamides, hydrocarbonsulphonates, hydrocarbyl sulphates, fatty acid salts, alkylpolyglycosides, ethylene oxide-propylene oxide block copolymers, and mixtures thereof.

18. The layered composite according to claim 17, wherein the ethylene oxide-propylene oxide block copolymers have a structure according to the formula (I): 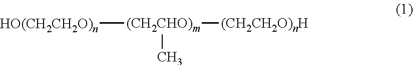
wherein n is from 2 to 200 and m is from 10 to 60.

19. The layered composite according to claim 16, wherein the aqueous, anionically hydrophilicized polyurethane dispersion is obtained by
A) providing isocyanate-functional prepolymers obtained from a reaction mixture comprising
A1) an organic polyisocyanate and
A2) a polymeric polyol having number average molecular weights from 400 to 8000 g/mol and OH functionalities from 1.5 to 6 and subsequently
B) reacting free NCO groups of the prepolymers in whole or in part with
B1) an amino-functional anionic or potentially anionic hydrophilicizing agent with chain extension and dispersing the isocyanate-functional prepolymers in water before, during or after B), wherein potentially ionic groups still present in the reaction mixture are converted into their ionic form by partial or complete reaction with a neutralizing agent.

20. The layered composite according to claim 19, wherein the reaction mixture in A) further comprises:
A3) hydroxyl-functional compounds having molecular weights of from 62 to 399 g/mol.

21. The layered composite according to claim 19, wherein the reaction mixture in A) further comprises:
A4) an isocyanate-reactive anionic or potentially anionic and optionally nonionic hydrophilicizing agent.

22. The layered composite according to claim 19, wherein free NCO groups of the isocyanate-functional prepolymers are further reacted in whole or in part in B) with B2) an amino-functional compound having a molecular weight from 32 to 400 g/mol.

23. The layered composite according to claim 19, wherein the organic polyisocyanate is selected from the group consisting of 1,6-hexamethylene diisocyanate, isophorone diisocyanate, isomeric bis(4,4′-isocyanatocyclohexyl)methanes, and mixtures thereof; and wherein the polymeric polyol comprises from 70 to 100% by weight of a mixture of polycarbonate polyols and polytetramethylene glycol polyols.

24. The layered composite according to claim 16, wherein the thermoplastic polymer of the cover layer comprises compounds selected from the group consisting of polyurethane, polyethylene, polypropylene, polyvinyl chloride, polystyrene, polyether, polyester, polyamide, polycarbonate, polyether-polyamide copolymers, polyacrylate, polymethacrylate, polymaleate, and mixtures thereof.

25. The layered composite according to claim 16, wherein the thermoplastic polymer of the cover layer comprises polyurethanes selected from the group consisting of aliphatic polyester polyurethanes, aromatic polyester polyurethanes, aliphatic polyether polyurethanes, aromatic polyether polyurethanes, and mixtures thereof.

26. The layered composite according to claim 16, wherein the direct bond between the foam layer and the cover layer has a peel strength from 0.01 to 0.50 N/mm.

27. The layered composite according to claim 16, wherein the layered composite has a water vapour permeability from 1000 to 4000 g/24 h×m².

28. A process for producing a layered composite according to claim 16, comprising the steps of:
providing a foam layer comprising a polyurethane foam obtained by frothing a composition comprising an aqueous, anionically hydrophilicized polyurethane dispersion;
applying a cover layer atop the foam layer.

29. The process according to claim 28, wherein the foam layer is dried, and wherein the cover layer is applied by laminating the cover layer atop the previously dried foam layer.

30. A wound dressing comprising the layered composite according to claim 16.

31. A sports article, textile article, or cosmetic article comprising the composite according to claim 16.
